(19) United States
(12) Patent Application Publication
(10) Pub. No.: US 2022/0387432 A1
(43) Pub. Date: Dec. 8, 2022

(54) COMPOSITIONS AND METHODS FOR TREATING PRION DISEASE

(71) Applicants: EIP Pharma, Inc., Boston, MA (US); Trustees of Boston University, Boston, MA (US)

(72) Inventors: John Jahangir Alam, Boston, MA (US); David A. Harris, Boston, MA (US); Cheng Fang, Boston, MA (US)

(73) Assignees: EIP Pharma, Inc., Boston, MA (US); Trustees of Boston University, Boston, MA (US)

(21) Appl. No.: 17/761,703

(22) PCT Filed: Sep. 18, 2020
(86) PCT No.: PCT/US20/51439

§ 371 Date: Mar. 18, 2022

Related U.S. Application Data
(60) Provisional application No. 62/902,959, filed on Sep. 19, 2019.

(51) Int. Cl. A61K 31/519; A61P 25/28

(52) U.S. Cl. CPC A61K 31/519; A61P 25/28

(57) ABSTRACT

The present invention provides methods and compositions for treatment of prion disease.

### CROSS-REFERENCE TO RELATED APPLICATIONS

This application claims priority to U.S. Provisional Application No. 62/902,959 filed Sep. 19, 2019, the entire content of which is incorporated by reference herein.

### GOVERNMENT SUPPORT

This invention was made with government support under NS065244 awarded by the National Institutes of Health. The United States government has certain rights in the invention.

### BACKGROUND

Prion diseases are infectious neurodegenerative diseases that affect humans and animals. The infectious agent, or prion, is composed of PrP^{Sc}, a conformationally altered form of a cell-surface glycoprotein designated PrP^{C}. Prions are thought to propagate by a templating process in which PrP^{Sc} molecules impose their β-sheet conformation on endogenous PrP^{C} substrate molecules.

### SUMMARY

The present disclosure encompasses the discovery that p38α mitogen activated protein kinase (MAPK) inhibitors can be used to inhibit or reverse neurodegenerative effects caused by prion disease. In particular, it has been found that administration of a p38α MAPK inhibitor can prevent or reverse retraction of dendritic spines caused by exposure to PrP^{Sc}.

In some embodiments, the disclosure provides methods of treating a subject having prion disease, comprising administering to the subject a p38α mitogen activated protein kinase (MAPK) inhibitor.

In some embodiments, the disclosure provides methods of inhibiting synaptic degeneration in a subject exposed to an infectious prion protein, comprising administering to the subject a p38α mitogen activated protein kinase (MAPK) inhibitor.

In some embodiments, the disclosure provides methods for preserving dendritic spines in a subject exposed to an infectious prion protein, comprising administering to the subject a p38α mitogen activated protein kinase (MAPK) inhibitor.

In some embodiments, the disclosure provides methods for reversing dendritic spine retraction in the central nervous system of a subject suffering from prion disease, comprising administering to the subject a p38α mitogen activated protein kinase (MAPK) inhibitor.

In some embodiments, the disclosure provides methods of restoring synaptic function in a subject exposed to an infectious prion protein, comprising administering to the subject a p38α mitogen activated protein kinase (MAPK) inhibitor.

In some embodiments, the p38α mitogen activated protein kinase (MAPK) inhibitor has greater affinity for isoform p38α than for isoforms p38β, p38δ, or p38γ. In some embodiments, the p38α mitogen activated protein kinase (MAPK) inhibitor is selective for the p38α isoform of p38 MAPK. In some embodiments, the p38α MAPK inhibitor is neflamapimod.

In some embodiments, the subject to be treated harbors a prion protein comprising PrP^{Sc}. In some embodiments, afflicted neuronal cells of the subject express an endogenous PrP^{C} protein. In some embodiments, the PrP^{C} protein comprises an amino acid sequence of KKRPKPGGW (SEQ ID NO: 3).

### BRIEF DESCRIPTION OF THE DRAWINGS

FIGS. 1A-1E show the effect of a p38 MAPK inhibitor on PrP^{Sc}-induced synaptotoxicity on hippocampal neurons in culture. Hippocampal neurons were treated for 24 hrs with purified PrP^{Sc} (FIGS. 1A-1C). One set of cultures (FIG. 1A) was then fixed and stained with fluorescent phalloidin; a second and third set were treated with PrP^{Sc} for an additional 24 hrs in the presence of vehicle (FIG. 1B) or a p38 MAPK inhibitor (SB239063, 10 μM) (FIG. 1C). A fourth set of cultures (FIG. 1D) was treated for 24 hrs with mock-purified material. All cultures were then fixed and stained with phalloidin. Pooled measurements of dendritic spine number were collected from 15±20 cells from 3 independent experiments (FIG. 1E). ***p<0.001 by Student's t-test; N.S., not significantly different. Scale bar in FIG. 1D=20 μm, also applicable to FIGS. 1A-1C.

FIGS. 2A-2F show the effect of a dominant-negative mutant of p38α MAPK (T180A/Y182F, referred to as p38AF) prevents PrP^{Sc}-induced synaptotoxicity. Hippocampal neurons from wild-type (WT) mice (FIGS. 2A and 2D) or p38AF dominant-negative (DN) mice (FIGS. 2B, 2C and 2E) were untreated (FIGS. 2A-2B), or were exposed to purified PrP^{Sc} (FIGS. 2D-2E) or mock purified material (FIG. C) for 24 hrs. Neurons were then fixed and stained with fluorescent phalloidin to visualize dendritic spines. The boxed regions in each panel are shown at higher magnification in the smaller panels to the right (FIGS. 2A-2C) and bottom (FIGS. 2D-2E). Arrowheads in the higher magnification panels in FIG. 2D show the positions of collapsed spines. Pooled measurements of spine number were collected from 15±20 cells from 4 animals (FIG. 2F). ***p<0.001 by Student's t-test; N.S., not significantly different. Scale bars in FIG. 2A=20 μm (main image) and 2 μm (higher magnification image), also applicable to FIGS. 2B-2E.

FIGS. 3A-3G show the effects of a selective p38α MAPK inhibitor, neflamapimod (also known as VX-745) on dendritic spines exposed to PrP^{Sc}. Hippocampal neurons were treated for 24 hrs with mock-purified material (FIG. 3A), purified PrP^{Sc} (FIG. 3B), or purified PrP^{Sc} in the presence of a p38α MAPK inhibitor (VX-745, 100 nM) (FIG. 3C). Dendritic spines were then visualized by fluorescent phalloidin staining (FIGS. 3A-3C). Pooled measurements of spine number were collected from 15-20 cells from 3 independent experiments (FIG. 3D). The bar labeled p38αi represents cultures treated with inhibitor without PrP^{Sc}. Parallel cultures were analyzed by patch clamping to measure mEPSC frequency and amplitude (FIGS. 3E-3G). N=10 cells from 2 independent experiments. ***p<0.001 and *p<0.05 by Student's t-test; N.S., not significantly different. Scale bar in FIG. 3A=20 μm, also applicable to FIGS. 3B-3C.

FIGS. 4A-4C show the effects of selective p38α MAPK inhibitor, neflamapimod (also known as VX-745) on PrP^{Sc} synaptotoxicity. (FIG. 4A) Primary hippocampal neuron cultures were treated with PrP^{Sc} and VX-745 (from 0 to 500 nM, as indicated) or with mock-purified material (last panel). Neurons were fixed after 24 hr of treatment and stained with Alexa488-labeled phalloidin for detection of F-actin, which is enriched in dendritic spines. (FIG. 4B) Quantification of spine number (per μm). (FIG. 4C) Dose response curve for the rescuing effect of VX-745, with a calculated EC₅₀=28.9 nM.

### DEFINITIONS

Carrier: The term “carrier” refers to any chemical entity that can be incorporated into a composition containing an active agent (e.g., a p38 MAPKα inhibitor) without significantly interfering with the stability and/or activity of the agent (e.g., with a biological activity of the agent). In certain embodiments, the term “carrier” refers to a pharmaceutically acceptable carrier.

Formulation: The term “formulation” as used herein refers to a composition that includes at least one active agent (e.g., p38 MAPKα inhibitor) together with one or more carriers, excipients or other pharmaceutical additives for administration to a patient. In general, particular carriers, excipients and/or other pharmaceutical additives are selected in accordance with knowledge in the art to achieve a desired stability, release, distribution and/or activity of active agent(s) and which are appropriate for the particular route of administration.

Pharmaceutically acceptable carrier, adjuvant, or vehicle: The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Therapeutically effective amount and effective amount: As used herein, and unless otherwise specified, the terms “therapeutically effective amount” and “effective amount” of an agent refer to an amount sufficient to provide a therapeutic benefit in the treatment, prevention and/or management of a disease, disorder, or condition, e.g., to delay onset of or minimize (e.g., reduce the incidence and/or magnitude of) one or more symptoms associated with the disease, disorder or condition to be treated. In some embodiments, a composition may be said to contain a “therapeutically effective amount” of an agent if it contains an amount that is effective when administered as a single dose within the context of a therapeutic regimen. In some embodiments, a therapeutically effective amount is an amount that, when administered as part of a dosing regimen, is statistically likely to delay onset of or minimize (reduce the incidence and/or magnitude of) one or more symptoms or side effects of a disease, disorder or condition.

Treat or Treating: The terms “treat” or “treating,” as used herein, refer to partially or completely alleviating, inhibiting, delaying onset of, reducing the incidence of, yielding prophylaxis of, ameliorating and/or relieving or reversing a disorder, disease, or condition, or one or more symptoms or manifestations of the disorder, disease or condition.

Unit Dose: The expression “unit dose” as used herein refers to a physically discrete unit of a formulation appropriate for a subject to be treated (e.g., for a single dose); each unit containing a predetermined quantity of an active agent selected to produce a desired therapeutic effect when administered according to a therapeutic regimen (it being understood that multiple doses may be required to achieve a desired or optimum effect), optionally together with a pharmaceutically acceptable carrier, which may be provided in a predetermined amount. The unit dose may be, for example, a volume of liquid (e.g., an acceptable carrier) containing a predetermined quantity of one or more therapeutic agents, a predetermined amount of one or more therapeutic agents in solid form (e.g., a tablet or capsule), a sustained release formulation or drug delivery device containing a predetermined amount of one or more therapeutic agents, etc. It will be appreciated that a unit dose may contain a variety of components in addition to the therapeutic agent(s). For example, acceptable carriers (e.g., pharmaceutically acceptable carriers), diluents, stabilizers, buffers, preservatives, etc., may be included. It will be understood, however, that the total daily usage of a formulation of the present invention will be decided by the attending physician within the scope of sound medical judgment. The specific effective dose level for any particular subject may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts. In some embodiments, a unit dose of a p38 MAPKα inhibitor is about 1 mg, 3 mg, 5 mg, 10 mg, 15 mg, 20 mg, 25 mg, 30 mg, 35 mg, 40 mg, 45 mg, 50 mg, 60mg, 80 mg, 100 mg, 125 mg, or 250 mg.

### DETAILED DESCRIPTION OF CERTAIN EMBODIMENTS

The present disclosure provides, among other things, compositions and methods for treating prion disease and associated pathology, by administering a composition comprising a p38 MAPKα inhibitor. In some embodiments, the p38α MAPK inhibitor is a selective p38α MAPK inhibitor. In some embodiments, the p38α MAP inhibitor is neflamapimod.

In some embodiments, the disclosure provides compositions and methods for treating subjects susceptible to or at risk of developing prion disease. In some embodiments, the disclosure provides compositions and methods for treating subjects exposed to an infectious prion protein.

Various aspects of the disclosure are described in detail in the following sections. The use of sections is not meant to limit the disclosure. Each section can apply to any aspect of the disclosure.

### Prion Disease

Prion diseases are a group of fatal, infectious neurodegenerative diseases affecting humans and animals. The infectious agent in prion disease is composed of PrP^{Sc}, a conformationally altered form of PrP^{C}. PrP^{C} is a (Glycosylphosphatidylinositol) GPI-anchored, cell surface glycoprotein that is widely expressed on neurons throughout the central nervous system beginning early in development.

Prions propagate by a particular templating process in which PrP^{Sc} molecules impose their unique, β-sheet-rich conformations on endogenous PrP^{C} substrate molecules. PrP knockout mice, in which PrP^{C} expression is absent, are completely resistant to prion infection. Moreover, these mice do not display symptoms of prion disease, indicating that the disease phenotype is primarily attributable to a specific function of PrP^{Sc}, rather than a loss of function of PrP^{C}. Neuropathological and imaging studies of infected mice suggest that synaptic degeneration begins early in the disease process before other pathological changes.

### P38 MAPK

Many extracellular stimuli, including pro-inflammatory cytokines and other inflammatory mediators, elicit specific cellular responses through the activation of mitogen-activated protein kinase (MAPK) signaling pathways. MAPKs are proline-targeted serine-threonine kinases that transduce environmental stimuli to the nucleus. Once activated, MAPKs activate other kinases or nuclear proteins through phosphorylation, including potential transcription factors and substrates. The four isoforms (α, β, δ, and γ) of p38 MAP kinase comprise one specific family of MAPKs in mammals that mediate responses to cellular stresses and inflammatory signals.

Pharmacological inhibitors of p38 MAPK have been developed as potential therapeutics for a variety of disorders. These include compounds that inhibit α, β, γ, δ isoforms of p38 MAPK (pan inhibitors), such as SB239063, compounds that inhibit both α and β isoforms such as RWJ67657, and compounds that selectively inhibit the a isoform, such as neflamapimod (VX-745) and BMS582949 (for review, see Shahin et al., (2017) Future Sci OA, 3(4) FS0204).

In some experimental paradigms, the pharmacological effects of pan inhibitors are distinguishable from those of isoform selective inhibitors. For example, in hippocampal cell culture, the pan p38 MAPK inhibitor SB239063 was found to be ineffective against amyloid β-derived diffusible ligand (ADDL) induced synaptotoxicity, whereas neflamapimod, a p38α selective MAPK inhibitor, showed positive effects (see Fang et al. PLoS (2018), 1-32, Amin et al., “Role of p38α MAP kinase in amyloid-β derived diffusible ligand (ADDL) induced dendritic spine loss in hippocampal neurons,” Alzheimer's Association International Conference, July 2019). However, in addition to inhibition of p38 MAPK, SB239063 has also been reported to inhibit casein kinase isoforms CKIδ and CKIε (Verkaar et al. (2011) Chem. & Biol.,18:485-494).

Pharmacological agents have been used to further explore synaptotoxic signaling pathways activated by PrP^{Sc} and Aβ oligomers. It has been reported that the ability of Aβ oligomers to cause dendritic spine retraction was blocked by the mGluR5 inhibitor, MPEP; but that MPEP had no influence on PrP^{Sc}-induced retraction of dendritic spines (Fang et al. PLoS (2018), 1-32). Moreover, a p38 MAPK inhibitor SB239063, which blocked PrP^{Sc}-induced synaptotoxicity, had no significant effect on Aβ oligomer induced dendritic spine loss. These data suggest that Aβ oligomers and PrP^{Sc} trigger different neurotoxic signaling pathways.

### Neflamapimod

Neflamapimod is a selective small-molecule inhibitor of the alpha isoform of p38 MAPK.

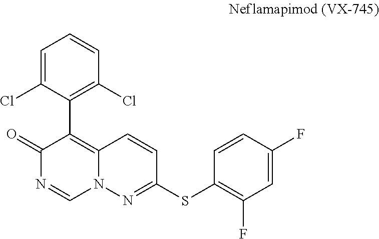 [SMILES: O=c1ncn2nc(Sc3ccc(F)cc3F)ccc2c1-c1c(Cl)cccc1Cl]

### Pharmaceutical Compositions

In some embodiments, a provided method comprises administering to a patient a pharmaceutical composition comprising a p38α MAPK inhibitor, such as neflamapimod, together with one or more therapeutic agents and a pharmaceutically acceptable carrier, adjuvant, or vehicle. In some embodiments, the present invention provides a pharmaceutical composition comprising a dose of p38α MAPK inhibitor together with one or more therapeutic agents and a pharmaceutically acceptable carrier, adjuvant, or vehicle, wherein the dose of p38α MAPK inhibitor results in an average blood concentration of from about 1 ng/mL to about 15 ng/mL, from about 1 ng/mL to about 10 ng/mL, from about 5 ng/mL to about 15 ng/mL, or from about 5 ng/mL to about 10 ng/mL.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Dosing

In some embodiments, compositions are administered in a therapeutically effective amount and/or according to a dosing regimen that is correlated with a particular desired outcome (e.g., with treating or reducing risk for disease).

In some embodiments, provided compositions are administered in a therapeutically effective amount and/or according to a dosing regimen that is correlated with a particular desired outcome (e.g., reduction in pathophysiology and/or symptoms of prion disease, etc.).

Alternatively or additionally, in some embodiments, an appropriate dose or amount is determined through use of one or more in vitro or in vivo assays to help identify desirable or optimal dosage ranges or amounts to be administered.

In various embodiments, provided compositions are administered at a therapeutically effective amount. As used herein, the term “therapeutically effective amount” or “therapeutically effective dosage amount” is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating the underlying disease or condition).

In some embodiments, a composition is provided as a pharmaceutical formulation. In some embodiments, a pharmaceutical formulation is or comprises a unit dose amount for administration in accordance with a dosing regimen correlated with achievement of disease reduction in symptoms of prion disease, arrest or decrease in rate of decline of function due to prion disease.

In some embodiments, a formulation comprising provided compositions as described herein is administered as a single dose. In some embodiments, a formulation comprising provided compositions as described herein is administered as two doses. In some embodiments, a formulation comprising provided compositions as described herein is administered at regular intervals. Administration at an “interval,” as used herein, indicates that the therapeutically effective amount is administered periodically (as distinguished from a one-time dose). The interval can be determined by standard clinical techniques. In some embodiments, a formulation comprising provided compositions as described herein is administered twice weekly, thrice weekly, every other day, daily, twice daily, or every eight hours.

In some embodiments, a formulation comprising provided compositions as described herein is administered once daily. In some embodiments, a formulation comprising provided compositions as described herein is administered twice daily. In some embodiments, the twice daily administering occurs from about 9 to 15 hours apart. In some embodiments the twice daily administering occurs about 12 hours apart. In some embodiments, a formulation comprising provided compositions as described herein is administered three times daily. In some embodiments, the three times daily administering occurs from about 4 to 8 hours apart. In some embodiments, the three times daily administering occurs 8 hours apart. In some embodiments, a formulation comprising from about 40 mg to about 250 mg of neflamapimod is administered twice daily. In some embodiments, the administering occurs when the patient is in a fed state. In some embodiments, the administering occurs within 30 to 60 minutes after the subject has consumed food. In some embodiments, the administering occurs when the patient is in a fasted state. The administration interval for a single individual need not be a fixed interval, but can be varied over time, depending on the needs of the individual.

In some embodiments, a formulation comprising provided compositions as described herein is administered at regular intervals. In some embodiments, a formulation comprising provided compositions as described herein is administered at regular intervals for a defined period. In some embodiments, a formulation comprising provided compositions as described herein is administered at regular intervals for 2 years, 1 year, 11 months, 10 months, 9 months, 8 months, 7 months, 6 months, 5 months, 4 months, 3 months, 2 months, a month, 3 weeks, 2, weeks, a week, 6 days, 5 days, 4 days, 3 days, 2 days or a day. In some embodiments, a formulation comprising provided compositions as described herein is administered at regular intervals for 16 weeks.

### Exemplification

The following examples are provided for illustrative purposes and are not intended to limit the scope of the invention.

### Materials and Methods

All procedures involving animals were conducted according to the United States Department of Agriculture Animal Welfare Act and the National Institutes of Health Policy on Humane Care and Use of Laboratory Animals.

### Low-Density Neuronal Cultures (Used for Dendritic Spine Measurements)

Timed-pregnant C57BL/6 mice (referred to as wild-type, WT) were purchased from the Jackson Laboratory (Bar Harbor, Me.). Prnp0/0 mice on a C57BL6 background were obtained from the European Mouse Mutant Archive (EMMA; Rome, Italy), and were maintained in a homozygous state by interbreeding.

Mice carrying a p38AF dominant-negative mutation on a C57BL6 background were obtained from the Jackson Laboratory (B6.Cg-Mapk14^{tm1.1Dvb/J}; stock #012736). The mutant allele was maintained in a heterozygous state by breeding with C57BL6 inbred mice. PCR genotyping of tail DNA was performed as per information and protocols are provided by Jackson Laboratory using the following primers: 5′-TAG AGC CAG CCC CAC TTT AGT C-3′ (SEQ ID NO: 1) and 5′-GAA GAT GGA TTT TAA GCA TCC GT-3′ (SEQ ID NO: 2). The expected PCR products included a 328 bp band representing the dominant-negative allele, and a 195 bp band representing the WT allele.

Hippocampal neurons were cultured from P0 pups. Neurons were seeded at 75 cells/mm² on poly-L-lysine-treated coverslips, and after several hours the coverslips were inverted onto an astrocyte feeder layer and maintained in NB/B27 medium until used. The astrocyte feeder layer was generated using murine neural stem cells. Neurons were kept in culture for 18±21 days prior to PrP^{Sc} treatment.

### Dendritic Spine Quantitation

Hippocampal neurons cultured as described above were treated with purified PrP^{Sc} or control preparations for 24 hrs, followed by fixation in 4% paraformaldehyde and staining with either Alexa 488-phalloidin or rhodamine-phalloidin (ThermoFischer Scientific, Waltham, Mass.) to visualize dendritic spines, and anti-tubulin antibodies (Sigma-Aldrich, St. Louis, Mo.) to visualize axons and dendrites. Images were acquired using a Zeiss 880 or Zeiss 700 confocal microscope with a 63× objective (N.A.=1.4). The number of dendritic spines was determined using ImageJ software. Briefly, 2±3 isolated dendritic segments were chosen from each image, and the images adjusted using a threshold that had been optimized to include the outline of the spines but not non-specific signals. The number of spines was normalized to the measured length of the dendritic segment to give the number of spines/μm. For each experiment, 15±24 neurons from 3±4 individual experiments were imaged and quantitated.

Immunostaining was performed with the following primary antibodies and corresponding secondary antibodies: anti-gephyrin (Synaptic Systems, Woodland, Calif.; cat 147011, 1:500); anti-tau (Santa Cruz Biotechnology, Santa Cruz, Calif.; cat. Sc5587, 1:500); anti-GluR1 (Abcam, Cambridge, Mass.; cat. Ab31232, 1:500); anti-synaptophysin (Millipore Sigma, St Louis, Mo.; cat. S5768, 1:500). Quantitation of gephyrin, GluR1, and synaptophysin staining was performed using ImageJ to count the number of fluorescent puncta per μm along isolated dendritic segments (similar to the method described above to quantitate dendritic spine numbers after phalloidin staining).

### Electrophysiological Analysis Using Mixed Hippocampal/Glial Cultures

Hippocampal cultures used for electrophysiological recording were prepared using a procedure that differs from the one used to prepare cultures for dendritic spine imaging. Briefly, hippocampi from newborn pups of the indicated genotypes were dissected and treated with 0.25% trypsin at 37° C. for 12 min.

Cells were plated at a density of 65,000 cells/cm2 on poly-D-lysine-coated coverslips in DMEM medium with 10% F12 and 10% FBS. Recordings were made from hippocampal neurons cultured for 18±20 days and treated for 24 hrs with purified PrP^{Sc} or control preparations. Whole-cell patch clamp recordings were collected using standard techniques. Pipettes were pulled from borosilicate glass and polished to an open resistance of 2±5 megaohms. Experiments were conducted at room temperature with the following solutions: internal, 140 mMCs-glucuronate, 5 mM CsCl, 4 mM MgATP, 1 mM Na2GTP, 10 mM EGTA, and 10 mM HEPES (pH 7.4 with CsOH); external, 150 mM NaCl, 4 mM KCl, 2 mM CaCl2, 2 mM MgCl2, 10 mM glucose, and 10 mM HEPES (pH 7.4 with NaOH). Current signals were collected from a Multiclamp 700B amplifier (Molecular Devices, Sunnyvale, Calif.), digitized with a Digidata 1550A interface (Axon Instruments, Union City, Calif.), and saved to disc for analysis with PClamp 10 software. Miniature excitatory postsynaptic currents (mEPSCs) were recorded in the presence of TTX (1 Abcam, Cat. #ab120054) and picrotoxin (100 μM, Abcam, Cat. #ab120315). Miniature inhibitory postsynaptic currents (mIPSCs) were recorded in the presence of TTX (1 μM) and CNQX (20 μM, Abcam, Cat. #ab120044). Frequencies and amplitudes of the mEPSCs and mIPSCs were quantitated by Clampfit (Molecular Devices, CA).

### Purification of PrP^{Sc}

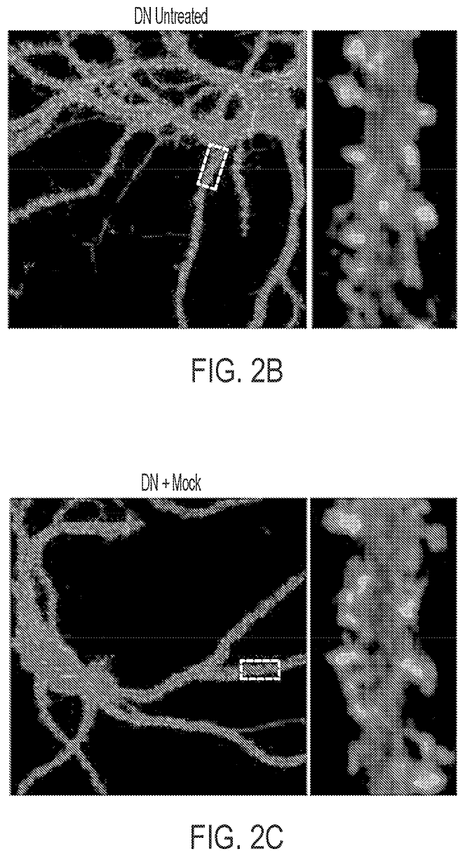

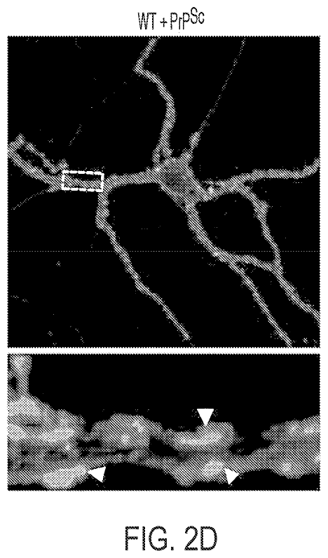

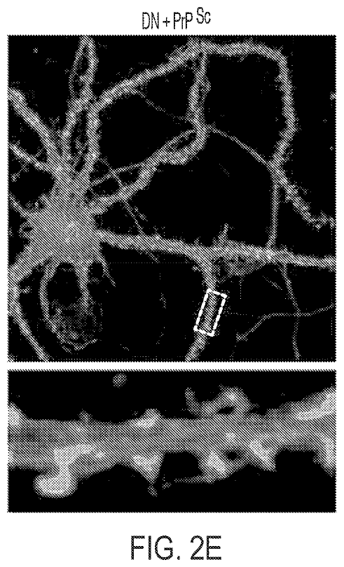

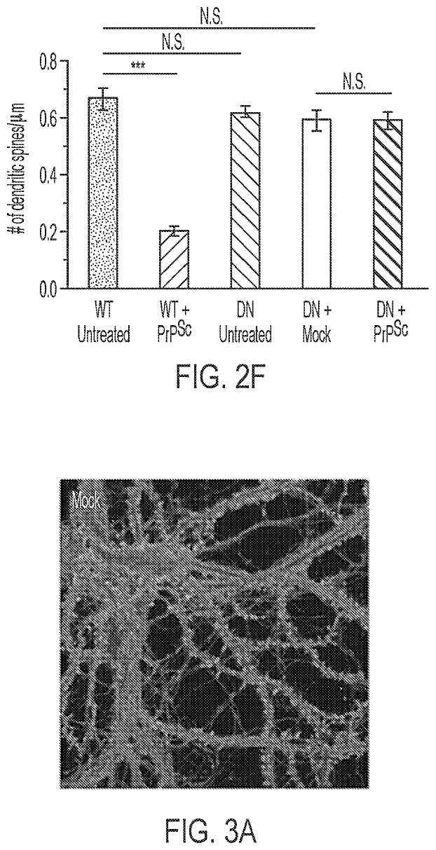

For the experiments shown in FIGS. 2A-2F, PrP^{Sc} was purified using a pronase E method based on the precipitation of PrP^{Sc} with sodium phosphotungstate (NaPTA) and limited proteolysis with pronase E. Brains were homogenized in PBS to generate a 10% (w/v) brain homogenate. After a clarification centrifugation step (500×g at 4° C. for 10 min), the supernatant was incubated with 2% sarkosyl for 1 hr and subsequently digested with 100 μg/ml of pronase E (Protease Type XIV from Streptomyces griseous; Sigma Aldrich, cat. no. P5147) for 30 min. The pronase E digestion was stopped with 2 mM PMSF and 10 mM EDTA. Afterwards, the samples were incubated with 0.3% (w/v) NaPTA (pH 7.0) for 1 hr and centrifuged at 16,000×g and 4° C. for 30 min. The pellet was resuspended in 2% sarkosyl and incubated overnight. The next day, the samples were adjusted to 0.3% (w/v) NaPTA and incubated for 1 hr, obtaining the final pellet by centrifugation at 18,000×g and 4° C. for 30 min. The final pellets were resuspended in PBS, with one brain-equivalent being resuspended in 50 μl of PBS. Aliquots were stored at −80° C. All digestions and incubations were performed at 37° C. with vigorous agitation.

For other experiments, PrP^{Sc} was purified as follows. Eighteen RML-infected C57BL6 brains were homogenized in 3 ml of 10% sarkosyl in TEND (10 mM Tris-HCl [pH 8], 1 mM EDTA, 130 mM NaCl, and 1 mM dithiothreitol) containing Complete Protease Inhibitor Cocktail (Roche Diagnostics, cat. no. 11836153001) using a glass bead homogenizer. Brain homogenates were incubated on ice for 1 hr and centrifuged at 22,000×g for 30 min at 4° C. The supernatant was kept on ice, while the pellet was resuspended in 1 ml of 10% sarkosyl in TEND, incubated for 1 hr on ice, and then centrifuged at 22,000×g for 30 min at 4° C. The pellet was discarded while the supernatants were pooled and centrifuged at 150,000×g for 2.5 h at 4° C. The new supernatants were discarded, while the pellets were rinsed with 50 ml of 100mM NaCl, 1% sulfobetaine (SB) 3±14 in TEND plus protease inhibitors, and then pooled by resuspending them in 1 ml of the wash buffer, and centrifuging at 180,000×g for 2 hr at 20° C. The supernatant was discarded, and the pellet was rinsed with 50 ml of TMS (10 mM Tris-HCl at pH 7.0, 5 mM MgCl2, and 100 mM NaCl) plus protease inhibitors, resuspended in 600 μl of the same buffer containing 100 mg/ml RNase A and incubated for 2 hr at 37° C. The sample was then incubated with 5 mM CaCl2, 20 mg/ml DNase I for 2 hr at 37° C. To stop the enzymatic digestion, EDTA was added to a final concentration of 20 mM, and the sample was mixed with an equal volume of TMS containing 1% SB 3±14. The sample was gently deposited on a 100 μl cushion of 1M sucrose, 100 mM NaCl, 0.5% SB 3±14, and 10 mM Tris-HCl (pH 7.4), and centrifuged at 180,000×g for 2 hr at 4° C. The supernatant was discarded and the pellet was rinsed with 50 μl of 0.5% SB 3±14 in PBS, resuspended in 1 ml of the same buffer, subjected to 5×5 sec pulses of bath sonication with a Bandelin Sonopuls Ultrasonicator (Amtrex Technologies, Montreal, Canada) at 90% power, and centrifuged at 180,000×g for 15 min at 4° C. The final supernatant was discarded and the final pellet was resuspended in 900 μl of PBS (50 μl for each starting brain) and sonicated 5 times for 5 sec. Aliquots were stored at −80° C. Mock purifications were also carried out from age-matched, uninfected brains. The purified preparations were evaluated by SDS-PAGE followed by silver staining and Western blotting.

Purified PrP^{Sc} was added to neuronal cultures at a final concentration of 4.4 μg/ml. An equivalent amount of mock material was used, based on purification from the same proportion of brain tissue.

### EXAMPLE 1

This example demonstrates the effects of p38 MAPK inhibition on PrP^{Sc}-induced synaptotoxicity. SB239063, which inhibits α, β, δ, and γ isoforms of P38 MAPK, prevented spine retraction caused by PrP^{Sc}.

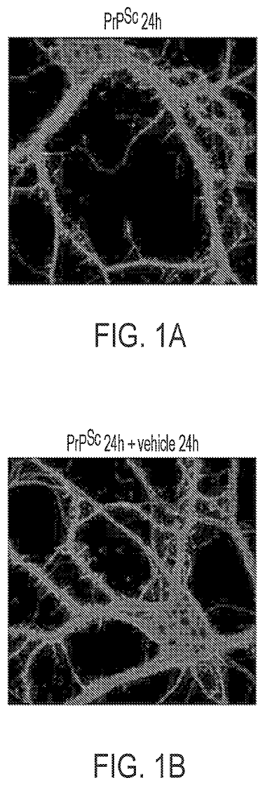

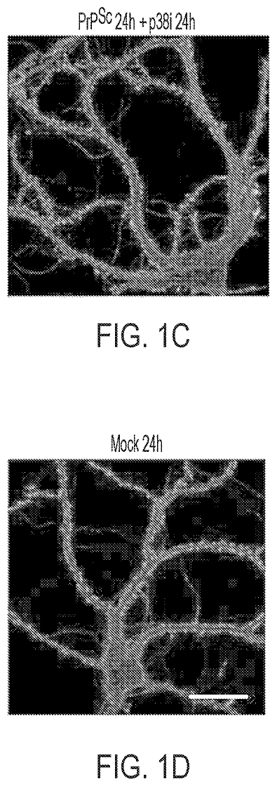

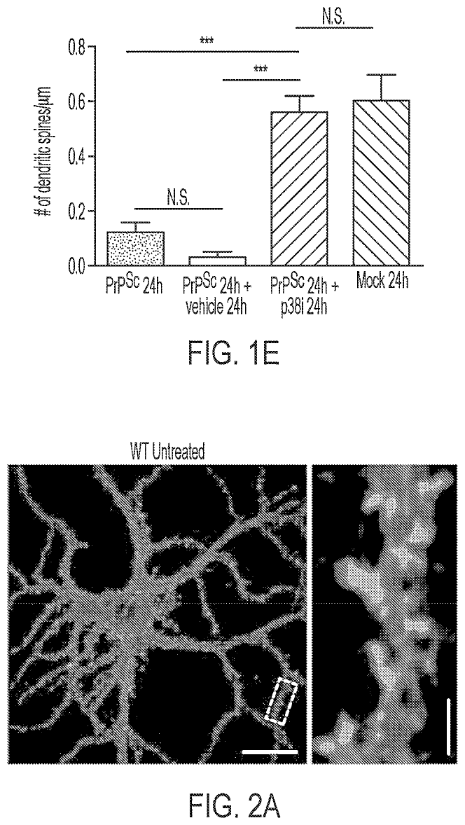

Neurons with PrP^{Sc} for 24 hrs, at which point most of the dendritic spines were retracted (FIG. 1A). Neurons were then exposed to PrP^{Sc} for an additional 24 hrs in the presence of a p38 MAPK inhibitor (SB239063) or vehicle control, after which cultures were fixed and assessed for dendritic spine morphology with fluorescent phalloidin. SB239063 was able to reverse the dendritic spine retraction that had accrued during the first 24 hrs of PrP^{Sc} treatment (FIG. 1C), compared to the cultures treated with vehicle (FIG. 1B). Quantitation of spine number under the three conditions is shown in FIG. 1E. These data indicate that the extensive dendritic spine abnormalities induced by PrP^{Sc} are reversible by p38 MAPK inhibition within a 48 hr time window.

### EXAMPLE 2

This example demonstrates a specific role for the p38α isoform of MAPK in PrP^{Sc}-induced synaptotoxcity.

A genetic method was employed to suppress signaling through the p38α MAPK pathway, which makes use of a dominant negative form of p38α MAPK (T180A/Y182F, referred to as p38AF). This double-mutation in the activation loop of the kinase prevents phosphorylation by upstream kinases, and has a dominant-negative effect on the activity of co-expressed wild-type p38, thereby significantly attenuating signaling. We prepared hippocampal neurons from mice that were heterozygous for the p38AF allele. This method of reducing p38 signaling avoids the embryonic lethal phenotype that results from complete germline inactivation of the p38 MAPK gene. It was found that neurons prepared from p38AF mice were morphologically comparable to WT neurons, but were almost completely resistant to the dendritic spine retraction effect of PrP^{Sc} (FIGS. 2A-2F).

### EXAMPLE 3

This example demonstrates that pharmacological inhibition of p38α MAPK can reverse PrP^{Sc}-induced synaptotoxicity. Administration of an inhibitor selective for the p38α isoform, neflamapimod (VX-745), blocked the effects PrP^{Sc} on dendritic spine number and mEPSC properties.

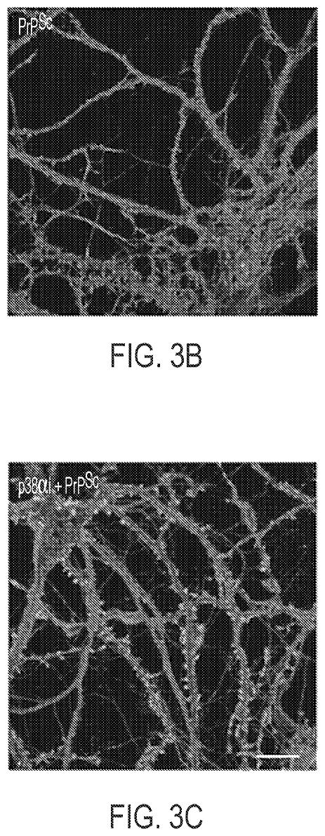

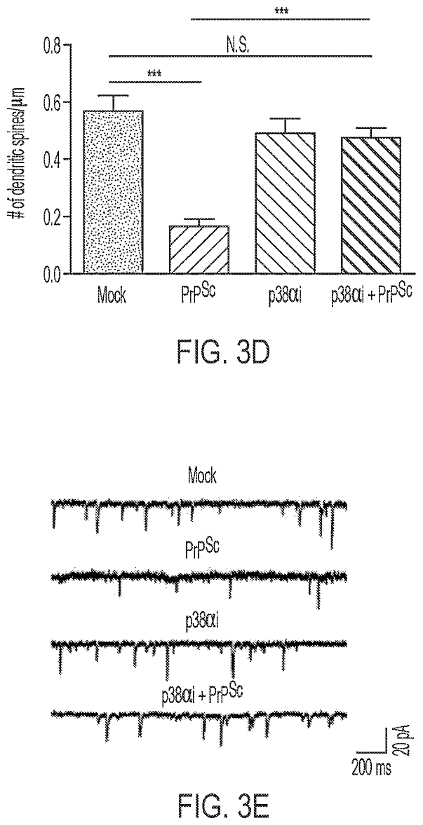

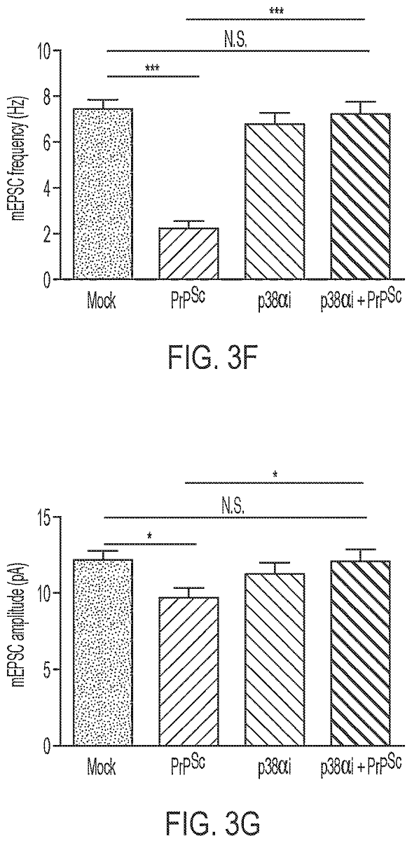

Hippocampal neurons were treated for 24 hrs with mock-purified material (FIG. 3A), purified PrP^{Sc} (FIG. 3B), or purified PrP^{Sc} in the presence of a p38α MAPK inhibitor (VX-745, 100 nM) (FIG. 3C). Dendritic spines were then visualized by fluorescent phalloidin staining (FIGS. 3A-3C). Pooled measurements of spine number were collected from 15-20 cells from 3 independent experiments (FIG. 3D). The bar labeled p38αi represents cultures treated with inhibitor without PrP^{Sc}. Parallel cultures were analyzed by patch clamping to measure mEPSC frequency and amplitude (FIGS. 3E-3G). N=10 cells from 2 independent experiments. ***p<0.001 and *p<0.05 by Student's t-test; N.S., not significantly different. Scale bar in FIGS. 3A-3G=20 μm.

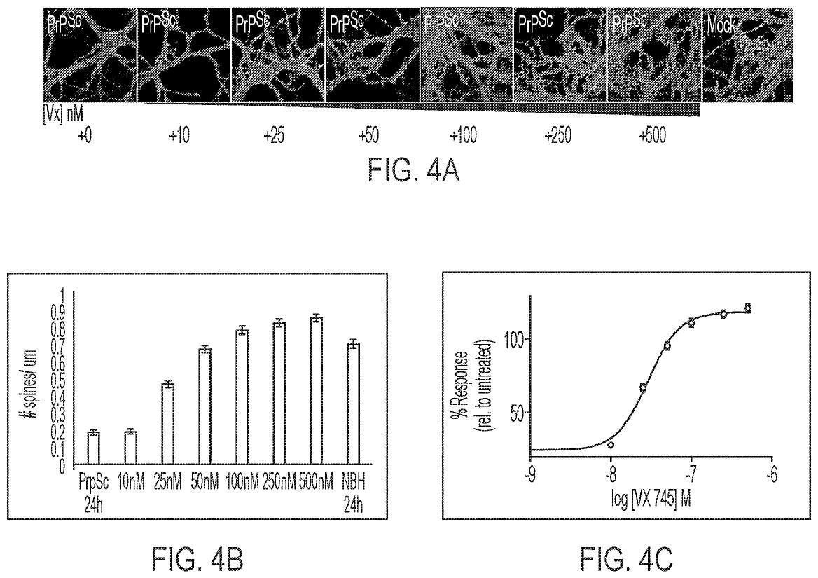

The effect of neflamapimod (VX-745) was demonstrated to be dose dependent. Primary hippocampal neuron cultures were treated with PrP^{Sc} and VX-745 (from 0 to 500 nM, as indicated in FIG. 4A) or with mock-purified material. Neurons were fixed after 24 hrs of treatment and stained with Alexa 488-labeled phalloidin for detection of F-actin, which is enriched in dendritic spines (FIG. 4A). Quantification of spine number (per μm) is shown in FIG. 4B. A dose response curve for the effect of VX-745 is shown in FIG. 4C, with a calculated EC₅₀ of 28.9 nM.

### EXAMPLE 4

Neflamapimod is administered to human subjects having prion disease or exposed to an infectious prion protein.

Neflamapimod 40 mg capsules are administered orally, BID or TID with food for 16 weeks; subjects will follow the BID regimen if weighing <80 kg or the TID regimen if weighing ≥80 kg. A placebo comparator is 40 mg matching placebo capsules administered orally, BID or TID with food for 16 weeks; subjects will follow the BID regimen if weighing <80 kg or the TID regimen if weighing ≥80 kg. Human subjects administered neflamapimod are examined for improvement in one or more symptoms of prion disease by clinical tests and/or restoration of synaptic integrity by brain imaging.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A method of treating a subject having prion disease, the method comprising administering to the subject a p38α mitogen activated protein kinase (MAPK) inhibitor.

2. A method of inhibiting synaptic degeneration in a subject exposed to an infectious prion protein, the method comprising administering to the subject a p38α mitogen activated protein kinase (MAPK) inhibitor.

3. A method for preserving dendritic spines in a subject exposed to an infectious prion protein, the method comprising administering to the subject a p38α mitogen activated protein kinase (MAPK) inhibitor.

4. A method for reversing dendritic spine retraction in the central nervous system of a subject suffering from prion disease, the method comprising administering to the subject a p38α mitogen activated protein kinase (MAPK) inhibitor.

5. A method of restoring synaptic function in a subject exposed to an infectious prion protein, the method comprising administering to the subject a p38α mitogen activated protein kinase (MAPK) inhibitor.

6. The method of any of claims 1-5, wherein the p38α mitogen activated protein kinase (MAPK) inhibitor has greater affinity for isoform p38α than for isoforms p38β, p38δ, or p38γ.

7. The method of any of claims 1-5 wherein the p38α mitogen activated protein kinase (MAPK) inhibitor is selective for the p38α isoform of p38 MAPK.

8. The method of any of claims 1-5, wherein the p38α MAPK inhibitor is neflamapimod.

9. The method of any of claims 1-8, wherein the prion protein comprises PrP^{Sc}.

10. The method of any of claims 1-9 wherein afflicted neuronal cells of the subject express an endogenous PrP^{C} protein.

11. The method of claim 10, wherein the PrP^{C} protein comprises an amino acid sequence of KKRPKPGGW (SEQ ID NO: 3).


SEQUENCE LISTING (3 sequences)

SEQ ID NO: 1 - DNA, 22 bp, Artificial Sequence
  Synthetic
tagagccagccccactttagtc

SEQ ID NO: 2 - DNA, 23 bp, Artificial Sequence
  Synthetic
gaagatggattttaagcatccgt

SEQ ID NO: 3 - protein, 9 aa, Artificial Sequence
  Synthetic
KKRPKPGGW